(19) United States
(12) Patent Application Publication
(10) Pub. No.: US 2011/0201550 A1
(43) Pub. Date: Aug. 18, 2011

(54) COMBINATION OF AMIDINE DERIVATIVES WITH CYCLIC DEPSIPEPTIDES

(75) Inventors: Achim Harder, Koeln (DE); Dittmar Nerger, Shanghai (CN); Thomas Bach, Wuppertal (DE); Venkata-Rangarao Kanikanti, Leverkusen (DE); Richard Kujanek, Koeln (DE)

(73) Assignee: Bayer Animal Health GMBH, Leverkusen (DE)

(21) Appl. No.: 13/000,510

(22) PCT Filed: Jun. 16, 2009
(86) PCT No.: PCT/EP2009/004304

§ 371 Date: Mar. 30, 2011

(30) Foreign Application Priority Data
Jun. 28, 2008 (DE) 102008030764.5

(51) Int. Cl. A61K 38/15; A61P 33/00

(52) U.S. Cl. 514/4.6

(57) ABSTRACT

The present invention relates to the combination of aminophenylamidine derivatives with cyclic depsipeptides, to products comprising this combination, and to the use of these active substances in combination for controlling endoparasites in humans and in animals.

The present invention relates to the combination of aminophenylamidine derivatives with cyclic depsipeptides, to products comprising this combination, and to the use of these active substances in combination for controlling endoparasites in humans and in animals.

Anthelmintically active aminophenylamidines and related compounds have been known for a long time, see, for example, DE OS 2 029 297, DE OS 2 029 298, DE OS 2 029 299 and DE OS 2 145 807. An important representative of this class is amidantel, which is known as a potent anthelminthic for dogs (Wollweber H et al. Arzneimittelforschung/Drug Research 29 (1) 31-32; DE-OS-20 29 298). The use in humans against Ancylostoma duodenale is described in Rim H. J. et al. The Korean Journal of Parasitology 18 (1) 24-36.

Amidantel is deacylated in vivo fairly rapidly to give the corresponding free amine (Bay d 9216), which is also anthelmintically active (Thomlinson et al., European Journal of Pharmacology. 113 (1985) 255-262).

Tribendimidine, a symmetrical diamidine derivative of amindantel, has been known for approximately 20 years and is developed in China as a broad-spectrum anthelmintic for use in humans (see, for example, Ren, H. N. et al. Chin. J. Parasitol. Parasit. Dis. 5 (1987) 262-264; Keiser, J. et al. Antimicrob. Agents Chemother. 51 (2007) 1096-1098). The control of endoparasites in livestock is subject matter of our patent application DE Ref. 10 2007 061262, which is pending in parallel.

Cyclic depsipeptides and their endoparasiticidal activity are known: enniatins and other 18-membered cyclic depsipeptides (EP-A 644 883, EP-A 658 551, EP-A 669 343, WO 95/27498); 24-membered cyclic depsipeptides (EP-A 626 376, EP-A 626 375, EP 787 141, EP-A 903 347, EP-A 973 756, WO 98/55469, WO 99/47506, WO 00/14079, WO 98/37088, WO 99/67281), cyclic depsipeptides with 12 ring atoms (EP-A 664 297). Cyclic octadepsipeptides such as PF1022 and emodepside and their activity against endoparasites (for example against intestinal nematodes and tissue nematodes) have also already been disclosed; see, for example EP-A 382 173, EP-A 634 408.

The present invention relates to:

products comprising anthelmintically active aminophenylamidine derivatives and cyclic depsipeptides.

Anthelmintically active aminophenylamidine derivatives are preferably compounds of the formula (I)

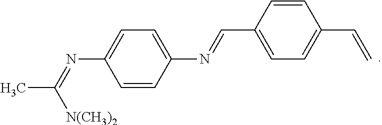

in which
R¹ is hydrogen or C₁₋₄-alkyl and
R² is hydrogen, —COO(C₁₋₄-alkyl), —CO(C₁₋₄-alkyl), —COCH₂(C₁₋₄-alkyl), —COCH₂O-phenyl or —CO-hetaryl, where hetaryl represents a 5- or 6-membered aromatic heterocycle with one or more hetero ring atoms selected from amongst O, N and S, or
R¹ and R² together represent the radical

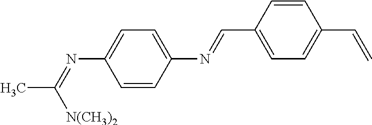

R¹ preferably represents hydrogen,
R² preferably represents hydrogen, —COCH₂(C₁₋₄-alkoxy) or together with R¹ represents the radical

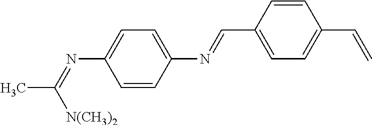

In accordance with a preferred embodiment, the anthelmintically active aminophenylamidine derivative is the compound amidantel, of the formula

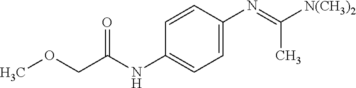

Amidantel and its preparation are described in DE-OS 2 029 298.

In accordance with a further preferred embodiment, the anthelmintically active aminophenylamidine derivative is the compound Bay d 9216, of the formula

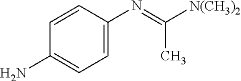

The preparation of N-(4-aminophenyl-N,N′-dimethylacetamidine (Bay d 9216) as precursor is also described in DE-OS 2 029 298.

In accordance with a further preferred embodiment, the anthelmintically active aminophenylamidine derivative is tribendimidine, of the formula

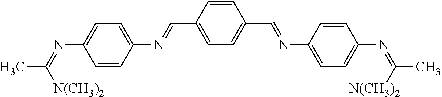

Tribendimidine and its synthesis are known. A preparation process is described for example, in Yao R H, Chen Y Q (1986) “Synthesis of Tribendimidine and its substituted analogues as new anthelmintic agents.” Annual Report of Institute of Parasitic Diseases, Chinese Academy of Preventive Medicine in 1986, pp. 199-202.

Depsipeptides are similar to peptides and differ from the latter by the fact that one or more α-amino acid units are replaced by α-hydroxycarboxylic acid units. Cyclic depsipeptides which are preferably employed in accordance with the invention are those with 18 to 24 ring atoms, in particular with 24 ring atoms (octadepsipeptides).

The depsipeptides with 18 ring atoms include compounds of the general formula (II):

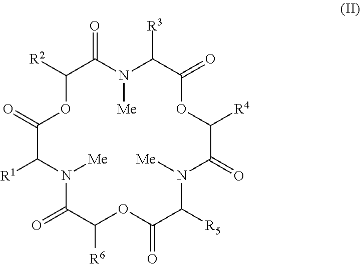

in which
R¹, R³ and R⁵ independently of one another represent hydrogen, straight-chain or branched alkyl having up to 8 carbon atoms, hydroxyalkyl, alkanoyloxyalkyl, alkoxyalkyl, aryloxyalkyl, mercaptoalkyl, alkylthioalkyl, alkylsulphinylalkyl, alkylsulphonylalkyl, carboxyalkyl, alkoxycarbonylalkyl, arylalkoxycarbonylalkyl, carbamoylalkyl, aminoalkyl, alkylaminoalkyl, dialkylaminoalkyl, guanidinoalkyl which can optionally be substituted by one or two benzyloxycarbonyl radicals or by one, two, three or four alkyl radicals, or represent alkoxycarbonylaminoalkyl, 9-fluorenylmethoxycarbonyl-(Fmoc)aminoalkyl, alkenyl, cycloalkyl, cycloalkylalkyl and optionally substituted arylalkyl, where halogen, hydroxyl, alkyl and alkoxy may be mentioned by way of substituents,
R², R⁴ and R⁶ independently of one another represent hydrogen, straight-chain or branched alkyl having up to 8 carbon atoms, hydroxyalkyl, mercaptoalkyl, alkanoyloxyalkyl, alkoxyalkyl, aryloxyalkyl, alkylthioalkyl, alkylsulphinylalkyl, alkylsulphonylalkyl, carboxyalkyl, alkoxycarbonylalkyl, arylalkoxycarbonylalkyl, carbamoylalkyl, aminoalkyl, alkylaminoalkyl, dialkylaminoalkyl, alkoxycarbonylaminoalkyl, alkenyl, cycloalkyl, cycloalkylalkyl, optionally substituted aryl or arylalkyl, where halogen, hydroxyl, alkyl, alkoxy may be mentioned by way of substituents, and their optical isomers and racemates.

Preferred compounds of the formula (II) are those in which

R¹, R³ and R⁵ independently of one another represent straight-chain or branched C₁-C₈-alkyl, in particular methyl, ethyl, propyl, isopropyl, butyl, isobutyl, sec-butyl, tert-butyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, mercapto-C₁-C₆-alkyl, in particular mercaptomethyl, C₁-C₄-alkylthio-C₁-C₆-alkyl, in particular methylthioethyl, C₁-C₄-alkylsulphinyl-C₁-C₆-alkyl, in particular methylsulphinylethyl, C₁-C₄-alkylsulfonyl-C₁-C₆-alkyl, in particular methylsulphonylethyl, carboxy-C₁-C₆-alkyl, in particular carboxymethyl, carboxyethyl, C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, carbamoyl-C₁-C₆-alkyl, in particular carbamoylmethyl, carbamoylethyl, amino-C₁-C₆-alkyl, in particular aminopropyl, aminobutyl, C₁-C₄-alkylamino-C₁-C₆-alkyl, in particular methylaminopropyl, methylaminobutyl, C₁-C₄-dialkylamino-C₁-C₆-alkyl, in particular dimethylaminopropyl, dimethylaminobutyl, guanido-C₁-C₆-alkyl, in particular guanidopropyl, C₁-C₄-alkoxycarbonylamino-C₁-C₆-alkyl, in particular tert-butoxycarbonylaminopropyl, tert-butoxycarbonyl-aminobutyl, 9-fluorenylmethoxycarbonyl(Fmoc)amino-C₁-C₆-alkyl, in particular 9-fluorenylmethoxy-carbonyl(Fmoc)aminopropyl, 9-fluorenylmethoxycarbonyl(Fmoc)aminobutyl, C₂-C₈-alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl-C₁-C₄-alkyl, in particular phenylmethyl which can optionally be substituted by radicals from the series consisting of halogen, in particular fluorine, chlorine, bromine or iodine, hydroxyl, C₁-C₄-alkoxy, in particular methoxy or ethoxy, C₁-C₄-alkyl, in particular methyl,
R², R⁴ and R⁶ independently of one another represent straight-chain or branched C₁-C₈-alkyl, in particular methyl, ethyl, propyl, isopropyl, butyl, isobutyl, sec-butyl, tert-butyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, mercapto-C₁-C₆-alkyl, in particular mercaptomethyl, C₁-C₄-alkylthio-C₁-C₆-alkyl, in particular methylthioethyl, C₁-C₄-alkylsulphinyl-C₁-C₆-alkyl, in particular methylsulphinylethyl, C₁-C₄-alkylsulfonyl-C₁-C₆-alkyl, in particular methylsulphonylethyl, carboxy-C₁-C₆-alkyl, in particular carboxymethyl, carboxyethyl, C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, carbamoyl-C₁-C₆-alkyl, in particular carbamoylmethyl, carbamoylethyl, amino-C₁-C₆-alkyl, in particular aminopropyl, aminobutyl, C₁-C₄-alkylamino-C₁-C₆-alkyl, in particular methylaminopropyl, methylaminobutyl, C₁-C₄-dialkylamino-C₁-C₆-alkyl, in particular dimethylaminopropyl, dimethylaminobutyl, C₂-C₈-alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl, phenyl-C₁-C₄-alkyl, in particular phenylmethyl which can optionally be substituted by radicals from the series consisting of halogen, in particular fluorine, chlorine, bromine or iodine, hydroxyl, C₁-C₄-alkoxy, in particular methoxy or ethoxy, C₁-C₄-alkyl, in particular methyl, and their optical isomers and racemates.

Especially preferred are compounds of the formula (II) in which

R¹, R³ and R⁵ independently of one another represent straight-chain or brandied C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, C₁-C₄-alkoxycarbonylamino-C₁-C₆-alkyl, in particular tert-butoxycarbonylaminopropyl, tert-butoxycarbonylaminobutyl, C₂-C₈-alkenyl, in particular vinyl, allyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl-C₁-C₄-alkyl, in particular phenylmethyl which can optionally be substituted by one or more identical or different radicals from among those mentioned above,
R², R⁴ and R⁶ independently of one another represent straight-chain or branched C₁-C₈-alkyl, in particular methyl, ethyl, propyl, isopropyl, butyl, isobutyl, sec-butyl, tert-butyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, aryl-C₁-C₄-allyloxy-C₁-C₆-alkyl, in particular benzyloxymethyl, 1-benzyloxyethyl, carboxy-C₁-C₆-alkyl, in particular carboxymethyl, carboxyethyl, C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, C₁-C₄-alkylamino-C₁-C₆-alkyl, in particular methylaminopropyl, methylaminobutyl, C₁-C₄-dialkyl-amino-C₁-C₆-alkyl, in particular dimethylaminopropyl, dimethylaminobutyl, C₂-C₈-alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl, phenyl-C₁-C₄-alkyl, in particular phenylmethyl which can optionally be substituted by one or more identical or different radicals from among those mentioned above, and their optical isomers and racemates.

Very especially preferred compounds of the formula (II) are those in which

R¹, R³ and R⁵ independently of one another represent straight-chain or branched C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, C₂-C₈-alkenyl, in particular allyl, C₃-C₇-cycloalkyl-C₁-C₄-alkyl, in particular cyclohexylmethyl, phenyl-C₁-C₄-alkyl, in particular phenylmethyl,
R², R⁴ and R⁶ independently of one another represent straight-chain or branched C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, C₂-C₈-alkenyl, in particular vinyl, allyl, C₃-C₇-cycloalkyl-C₁-C₄-alkyl, in particular cyclohexylmethyl, phenyl-C₁-C₄-alkyl, in particular phenylmethyl which can optionally be substituted by one or more identical or different radicals from among those mentioned above, and their optical isomers and racemates.

The following compounds of the general formula (II), in which the radicals R¹ to R⁶ have the following meanings, may be mentioned individually:

| R¹ | R² | R³ | R⁴ | R⁵ | R⁶ |
|---|---|---|---|---|---|
| —CHMeCH₂Me | —cyclohexyl | —CHMeCH₂Me | —Me | —CHMeCH₂Me | —Me |
| —CHMeCH₂Me | —cyclohexyl | —CHMeCH₂Me | —Me | —CHMeCH₂Me | —cyclohexyl |
| —CHMeCH₂Me | —CH₂-Phe | —CHMeCH₂Me | —Me | —CHMeCH₂Me | —Me |
| —CHMeCH₂Me | —CH₂-Phe | —CHMeCH₂Me | —Me | —CHMeCH₂Me | —CH₂-Phe |
| —CHMeCH₂Me | —(CH₂)₃—Me | —CHMeCH₂Me | —Me | —CHMeCH₂Me | —Me |
| —CHMeCH₂Me | —(CH₂)₃—Me | —CHMeCH₂Me | —Me | —CHMeCH₂Me | —(CH₂)₃—Me |
| —CHMe₂ | —CH₂-Phe | —CHMeCH₂Me | —Me | —CHMeCH₂Me | —Me |
| —CH₂-Phe | —CHMe₂ | —CH₂-Phe | —CHMe₂ | —CHMeCH₂Me | —CHMe₂ |
| —CH₂CHMe₂ | —CH₂-Phe | —CH₂CHMe₂ | —Me | —CH₂CHMe₂ | —CH₂-Phe |
| —(CH₂)₃—Me | —Me | —CHMeCH₂Me | —Me | —CHMeCH₂Me | —Me |
| —CHMe₂ | —Me | —CHMe₂ | —Me | —CHMe₂ | —Me |
| —CH₂—Me | —Me | —CH₂—Me | —Me | —CH₂—Me | —Me |
| —(CH₂)₂—Me | —Me | —(CH₂)₂—Me | —Me | —(CH₂)₂—Me | —Me |
| —(CH₂)₃—Me | —Me | —(CH₂)₃—Me | —Me | —(CH₂)₃—Me | —Me |
| —CH₂—CH═CH₂ | —Me | —CH₂—CH═CH₂ | —Me | —(CH₂)—CH═CH₂ | —Me |
| —CHMeCH₂Me | —Me | —CHMeCH₂Me | —Me | —CHMeCH₂Me | —CH₂—Me |
| —CHMeCH₂Me | —Me | —CHMeCH₂Me | —Me | —CHMeCH₂Me | —(CH₂)₂—Me |
| —CHMeCH₂Me | —Me | —CHMeCH₂Me | —Me | —CHMeCH₂Me | —(CH₂)₃—Me |
| —CHMeCH₂Me | —Me | —CHMeCH₂Me | —Me | —CH₂Me | —Me |
| —CHMeCH₂Me | —Me | —CHMeCH₂Me | —Me | —(CH₂)₂—Me | —Me |
| -cyclohexyl | —Me | -cyclohexyl | —Me | -cyclohexyl | —Me |
| —CH₂CHMe₂ | —cyclohexyl | —CH₂CHMe₂ | —Me | —CH₂CHMe₂ | —cyclohexyl |
| —CH₂CHMe₂ | —cyclohexyl | —CH₂CHMe₂ | —Me | —CH₂CHMe₂ | —Me |
| —CHMeCH₂Me | —CHMe₂ | —CHMeCH₂Me | —CHMe₂ | —CHMeCH₂Me | —Me |
| —CH₂-Phe | —Me | —CH₂-Phe | —Me | —CH₂-Phe | —Me |
| -cyclohexyl | —Me | -cyclohexyl | —Me | -cyclohexyl | —Me |
| —CHMe₂ | —CHMe₂ | —CHMe | —Me | —CHMe₂ | —Me |
| —CHMe₂ | —CHMe₂ | —CHMe₂ | —CHMe₂ | —CHMe₂ | —Me |
| —CH₂—Me | —CHMe₂ | —CH₂Me | —Me | —CH₂—Me | —Me |
| —CH₂—Me | —CHMe₂ | —CHMe₂ | —CHMe₂ | —CH₂—Me | —Me |
| —(CH₂)₂—Me | —CHMe₂ | —(CH₂)₂—Me | —Me | —(CH₂)₂—Me | —Me |
| —(CH₂)₂—Me | —CHMe₂ | —(CH₂)₂—Me | —CHMe₂ | —(CH₂)₂—Me | —Me |
| —(CH₂)₃—Me | —CHMe₂ | —(CH₂)₃—Me | —Me | —(CH₂)₃—Me | —Me |
| —(CH₂)₃—Me | —CHMe₂ | —(CH₂)₃—Me | —CHMe₂ | —(CH₂)₃—Me | —Me |
| —CH₂—CH═CH₂ | —CHMe₂ | —CH₂—CH═CH₂ | —Me | —CH₂—CH═CH₂ | —Me |
| —CH₂—CH═CH₂ | —CHMe₂ | —CH₂—CH═CH₂ | —CHMe₂ | —CH₂—CH═CH₂ | —Me |
| —Me | —Me | —CHMeCH₂Me | —Me | —CH₂—Me | —Me |
| —Me | —Me | —CHMeCH₂Me | —Me | —(CH₂)₃—Me | —Me |

Me = methyl;
Phe = phenyl

A further depsipeptide which may be mentioned is the compound PF 1022 of the formula (IIIa) hereinbelow, which is known from EP-A 382 173:

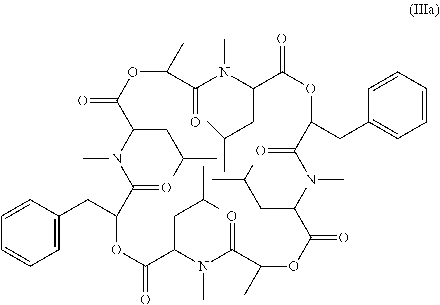

Further depsipeptides which may be mentioned are the compounds known from the PCT application WO 93/19053.

Compounds which may be mentioned in particular from WO 93/19053 are those of the following formula (IIIb):

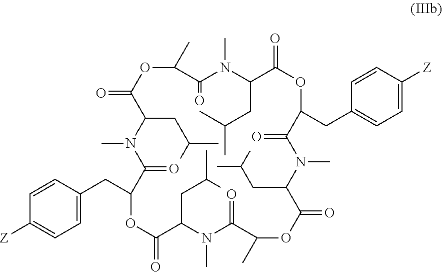

in which
Z represents N-morpholinyl, amino, mono- or dimethylamino.

Further compounds which may be mentioned are those of the following formula (IIIc):

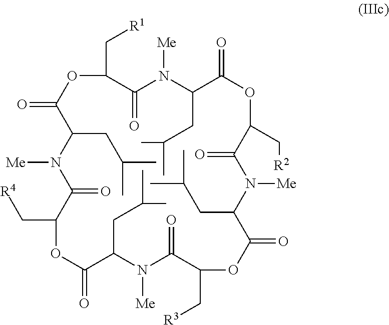

in which
R¹, R², R³, R⁴ independently of one another represent hydrogen, C₁-C₁₀-alkyl or aryl, in particular phenyl, which are optionally substituted by hydroxyl, C₁-C₁₀-alkoxy or halogen.

The compounds of the general formula (II) are known and can be obtained by the processes described in EP-A-382 173, DE-A 4 317 432, DE-A 4 317 457, DE-A 4 317 458, EP-A-634 408, EP-A-718 293, EP-A-872 481, EP-A-685 469, EP-A-626 375, EP-A-664 297, EP-A-669 343, EP-A-787 141, EP-A-865 498, EP-A-903 347.

The cyclic depsipeptides with 24 ring atoms also include compounds of the general formula (IIId)

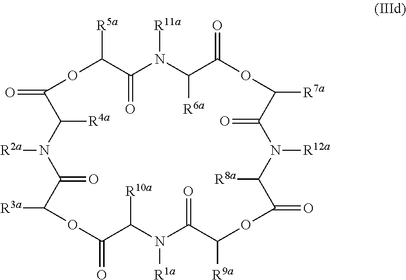

in which
R^{1a}, R^{2a}, R^{11a} and R^{12a} independently of one another represent C₁₋₈-alkyl, C₁₋₈-haloalkyl, C₃₋₆-cycloalkyl, aralkyl, aryl,
R^{3a}, R^{5a}, R^{7a}, R^{9a} independently of one another represent hydrogen or straight-chain or branched C₁₋₈-alkyl which can optionally be substituted by hydroxyl, C₁₋₄-alkoxy, carboxyl,

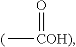

carboxamide,

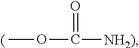

imidazolyl, indolyl, guanidino, —SH or C₁₋₄-alkylthio and which furthermore represents aryl or aralkyl, each of which can be substituted by halogen, hydroxyl, C₁₋₄-alkyl, C₁₋₄-alkoxy,
R^{4a}, R^{6a}, R^{8a}, R^{10a} independently of one another represent hydrogen; straight-chain C₁₋₅-alkyl, C₂₋₆-alkenyl, C₃₋₇-cycloalkyl, each of which can optionally be substituted by hydroxyl, C₁₋₄-alkoxy, carboxyl, carboxamide, imidazolyl, indolyl, guanidino, SH or C₁₋₄-alkylthio, and also represent aryl or aralkyl, each of which can be substituted by halogen, hydroxyl, C₁₋₄-alkyl, C₁₋₄-alkoxy; and their optical isomers and racemates.

It is preferred to employ compounds of the formula (IIId) in which

R^{1a}, R^{2a}, R^{11a} and R^{12a} independently of one another represent methyl, ethyl, propyl, isopropyl, n-, s-, t-butyl or phenyl which is optionally substituted by halogen, C₁₋₄-alkyl, OH, C₁₋₄-alkoxy, and also represent benzyl or phenylethyl, each of which can optionally be substituted by the radicals mentioned under phenyl;
R^{3a} to R^{10a} have the abovementioned meanings.

Especially preferred compounds are those of the formula (IIId) in which

R^{1a}, R^{2a}, R^{11a} and R^{12a} independently of one another represent methyl, ethyl, propyl, isopropyl or n-, s-, t-butyl,
R^{3a}, R^{5a}, R^{7a}, R^{9a} represent hydrogen, straight-chain or branched C₁₋₈-alkyl, in particular methyl, ethyl, propyl, isopropyl, n-, s-, t-butyl, each of which can optionally be substituted by C₁₋₄-alkoxy, in particular methoxy, ethoxy, imidazolyl, indolyl or C₁₋₄-alkylthio, in particular methylthio, ethylthio, and furthermore represent phenyl, benzyl or phenethyl, each of which can optionally be substituted by halogen, in particular chlorine.
R^{4a}, R^{6a}, R^{8a}, R^{10a} independently of one another represent hydrogen; methyl, ethyl, n-propyl, n-butyl, vinyl, cyclohexyl, each of which can optionally be substituted by methoxy, ethoxy, imidazolyl, indolyl, methylthio, ethylthio, and also represent isopropyl, s-butyl, and furthermore optionally halogen-substituted phenyl, benzyl or phenylethyl.

The compounds of the formula (IIId) can likewise be obtained by the processes described in EP-A-382 173, DE-A 4 317 432, DE-A 4 317 457, DE-A 4 317 458, EP-A-634 408, EP-A-718 293, EP-A-872 481, EP-A-685 469, EP-A-626 375, EP-A-664 297, EP-A-669 343, EP-A-787 141, EP-A-865 498, EP-A-903 347.

Depsipeptides which are very especially preferred in accordance with the invention are PF 1022 A (see formula (IIIa) and emodepside (PF 1022-221), compound of the formula (IIIb) in which both radicals Z represent the morpholinyl radical). The INN emodepside represents the compound with the systematic name: cyclo[(R)-lactoyl-N-methyl-L-leucyl-(R)-3-(p-morpholinophenyl)lactoyl-N-methyl-L-leucyl-(R)-lactoyl-N-methyl-L-leucyl-(R)-3-(p-morpholinophenyl)lactoyl-N-methyl-L-leucyl].

Depending on the structure, active substances can exist in stereoisomeric forms or as stereoisomer mixtures, for example as enantiomers or racemates. Not only the stereoisomer mixtures, but also the pure stereoisomers, can be used in accordance with the invention.

The following may optionally also be used: salts of the active substances with pharmaceutically acceptable acids or bases and also solvates, in particular hydrates, of the active substances or of their salts.

The active substances, which are used in the products according to the invention, may exist in stereoisomeric forms (enantiomers, diastereomers), depending on their structure. In accordance with the invention, it is possible to employ the enantiomers or diastereomers and their respective mixtures.

If the active substances can exist in tautomeric forms, the present invention also includes the use of the tautomeric forms.

If appropriate, the active substances may also be employed in the form of their salts, solvates and solvates of the salts.

Preferred in accordance with the present invention as salts are physiologically acceptable salts of the active substances.

Depending on the structure of the active substance, physiologically acceptable salts of the active substances comprise acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

If appropriate, physiologically acceptable salts of the active substances also comprise the salts of customary bases, such as, by way of example and by preference, alkali metal salts (for example sodium and potassium salts), alkaline earth metal salts (for example calcium and magnesium salts) and ammonium salts, derived from ammonia or organic amines having 1 to 16 C atoms, such as by way of example and by preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Within the context of the invention, the term solvates refers to those forms of the active substances which, in the solid or liquid state, form a complex by coordination with solvent molecules. Hydrates are a specific form of the solvates in which the coordination takes place with water.

Moreover, the present invention also relates to prodrugs of the active substances. The term “prodrugs” comprises compounds which themselves may be biologically active or inactive, but which, during their residence time in the body, are converted into the actual substance (for example metabolically or hydrolytically).

The products according to the invention, which have a favourable toxicity to warm blooded species, are suitable for controlling pathogenic endoparasites which are found in humans and in animal keeping and animal breeding in productive livestock, breed animals, zoo animals, laboratory animals, experimental animals and pets. They are active against all or individual developmental stages of the pests and against resistant and normally-sensitive species. By controlling the pathogenic endoparasites, it is intended to reduce disease, death and reduced performance (for example in the production of meat, milk, wool, hide, eggs, honey and the like), so that more economical and simpler animal keeping is possible by employing the active substances. The pathogenic endoparasites include cestodes, trematodes, nematodes, acantocephala, in particular:

From the order of the Pseudophyllidea, for example, Diphyllobothrium spp., Spirometra spp. Schistocephalus spp. Ligula spp. Bothridium spp., Diphlogonoporus spp.

From the order of the Cyclophyllidea, for example, Mesocestoides spp. Anoplocephala spp., Paranoplocephala spp., Moniezia spp., Thysanosomsa spp., Thysaniezia spp., Avitellina spp., Stilesia spp., Cittotaenia spp. Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

From the subclass of the Monogenea, for example, Gyrodactylus spp., Dactylogyrus spp., Polystoma spp.

From the subclass of the Digenea, for example, Diplostomum spp. Posthodiplostomum spp., Schistosoma spp., Trichohilharzia spp., Ornithohilharzia spp., Austrobilharzia spp., Gigantobilharzia spp. Leucochloridium spp. Brachylaima spp. Echinostoma spp. Echinoparyphium spp. Echinochasmus spp., Hypoderacum spp., Fasciola spp. Fasciolides spp., Fasciolopsis spp., Cyclocoelum spp., Typhlocoelum spp., Paramphistomum spp., Calicophoron spp. Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.

From the order of the Enoplida, for example, Trichuris spp., Capillaria spp., Trichlomosoides spp., Trichinella spp.

From the order of the Rhabditia, for example, Micronema spp., Strongyloides spp.

From the order of the Strongylida, for example, Strongylus spp., Triodontophorus spp., Oesophagodontus spp., Trichonema spp., Gyalocephalus spp., Cylindropharynx spp., Poteriostromum spp., Cyclococercus spp., Cylicostephanus spp., Oesophagostomum spp., Chabertia spp., Stephanurus spp., Ancylostoma spp. Uncinaria spp., Bunostomum spp., Globocephalus spp. Syngamus spp., Cyathostomum spp., Metastrongylus spp., Dictyocaulus spp., Muellerius spp. Protostrongylus spp. Neostrongylus spp., Cystocaulus spp., Pneumostrongylus spp. Spicocaulus spp., Elaphostrongylus spp., Parelaphostrongylus spp., Crenosoma spp., Paracrenosoma spp., Angiostrongylus spp., Aelurostrongylus spp., Filaroides spp. Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp. Cylicocyclus spp., Cylicodontophorus spp.

From the order of the Oxyurida, for example, Oxyuris spp., Enterobius spp., Passalurus spp., Syphacia spp., Aspiculuris spp. Heterakis spp.

From the order of the Ascaridia, for example, Ascaris spp., Toxascaris spp., Toxocara spp., Parascaris spp., Anisakis spp., Ascaridia spp.

From the order of the Spirurida, for example, Gnathostoma spp., Physaloptera spp., Thelazia spp. Gongylonema spp., Habronema spp., Parabronema spp. Draschia spp., Dracunculus spp.

From the order of the Filariida, for example, Stephanofilaria spp., Parafilaria spp. Setaria spp. Loa spp. Dirofilaria spp. Litomosoides spp., Brugia spp. Wuchereria spp. Onchocerca spp.

From the group of the Gigantohynchida, for example, Filicollis spp. Moniliformis spp., Macracanthorhynchus spp., Prosthenorchis spp.

Preferred is the control of tapeworms, for example, Taenia spp. Also preferred is the control of nematodes, such as, for example:

From the order of the Spirurida, for example, Gnathostoma spp., Physaloptera spp., Thelazia spp., Gongylonema spp., Habronema spp., Parabronema spp., Draschia spp., Dracunculus spp.

The use of the products according to the invention is especially preferred for the control of Strongylida, in particular Haemonchus spp., Trichostrongylus spp., Cooperia spp. and Ostertagia spp. and of Ascaridida such as, for example. Parascaris spp.

The productive livestock includes in particular mammals such as, for example, cattle, horses, sheep, pigs, goats, camels, water buffalos, donkeys, rabbits, fallow deer, reindeer, fur hearers such as, for example, mink, chinchilla, raccoon. Preferred among the mammalian productive livestock are cattle, sheep and pigs.

Also included for the use according to the invention are the following animal species, which are not mammals, but also belong to the productive livestock: birds such as, for example, chickens, geese, turkeys, ducks; fresh water and salt water fish such as, for example, trout, carp, eels, reptiles, insects such as, for example, honeybee and silkworm.

The pets preferably include dogs and cats. In these, it is preferred to control Toxoscaris spp., Toxocara spp., Trichuris spp. Trichinella spp. and the hookworms Ancylostoma spp. and Uncinaria spp.

In accordance with a further embodiment, the products may also be employed in humans. In humans, it is preferred to control Ascaris spp., Ancylostoma spp. Necator spp., Trichuris spp. Strongyloides spp. and Enterobius spp.

Among the mammals, it is the herbivores (plant eaters) which are preferred for the use of the abovementioned combinations, that is to say, animals which feed mainly on plants. The treatment of ruminants (such as, for example, sheep, goats, cattle) is particularly preferred.

Nonruminant herbivores which are mammals and which may be mentioned as a preferred example are horses. In horses, the abovementioned combinations can be employed preferably for example for controlling Strongylida or in particular roundworms (Ascaridia), such as, for example, Parascaris equorum.

Among the ruminants, it is preferably Strongylida, in particular Haemonchus spp., Trichostrongylus spp. Cooperia spp. and Ostertagia spp. which can be controlled.

In accordance with the invention, sheep are treated with particular preference.

In accordance with the invention, cattle are also treated with particular preference.

The application can be effected both prophylactically and therapeutically.

The active substance is applied directly or in the form of suitable preparations, either enterally, parenterally, dermally, nasally, by treating the environment, or with the aid of active-substance-containing shaped articles such as, for example, strips, tablets, tapes, collars, ear tags, limb bands, marking devices.

The enteral application of active substance is effected orally, for example in the form of powders, suppositories, tablets, capsules, pastes, drinks, granules, drenches, boluses, medicated feed or drinking water. It is applied dermally for example in the form of dipping, spraying, bathing, washing, pouring-on and spotting-on and dusting. It is administered parenterally for example in the form of an injection (intramuscular, subcutaneous, intravenous, intraperitoneal) or by implants.

Suitable preparations are:

solutions such as solutions for injection, oral solutions, concentrates for oral administration after dilution, solutions for use on the skin or in body cavities, pour-on and spot-on formulations, gels;
emulsions and suspensions for oral or dermal administration and for injection; semi-solid preparations;
formulations in which the active substance is incorporated into an ointment base or into an oil-in-water or water-in-oil emulsion base;
solid preparations such as powders, premixes or concentrates, granules, pellets, tablets, boluses, capsules; aerosols and inhalates, active-substance-containing shaped articles.

Solutions for injection are administered intravenously, intramuscularly and subcutaneously.

Oral solutions are employed directly. Concentrates are used orally after previously having been diluted to the use concentration.

Solutions for use on the skin are trickled on, brushed on, rubbed in, sprinkled on, sprayed on or applied by dipping, bathing or washing.

Gels are applied or brushed onto the skin or introduced into body cavities.

Pour-on and spot-on formulations are poured or sprinkled onto limited areas of the skin, the active substance either penetrating the skin and acting systematically or spreading over the body surface.

Emulsions can be employed orally, dermally or in the form of an injection. Emulsions are either of the water-in-oil type or of the oil-in-water type.

Suspensions can be employed orally, dermally or in the form of an injection.

Semi-solid preparations can be administered orally or dermally.

To prepare solid preparations, the active substance is mixed with suitable carriers, if appropriate, with addition of adjuvants, and shaped as desired.

If appropriate, the products according to the invention may contain further active substances.

The use in combination means either that the anthelmintically active aminophenylamidine derivatives and the cyclic depsipeptides are formulated in a combined preparation and, accordingly, applied jointly; however, the products can also comprise separate preparations for each active substance. In the event that more than two active substances are to be employed, all active substances can, accordingly, be formulated in combined preparations, or all active substances can be formulated in separate formulations; also feasible are mixed forms where some of the active substances are formulated together and some of the active substances are formulated separately.

Separate formulations permit the separate or staggered application of the active substances in question.

The products (preparations) contain the active substances in each case in concentrations of from 10 ppm to 90 percent by weight, preferably from 0.1 to 50 percent by weight.

Ready-to-use preparations usually contain the active substances in concentrations of from in each case 10 ppm to 20 percent by weight, preferably of from 0.1 to 10 percent by weight.

Preparations which are diluted prior to administration contain the active substances in concentrations of from 0.5-90% by weight, preferably of from 5 to 50 percent by weight.

The weight ratio of aminophenylamidine to cyclodepsipeptide in the products according to the invention depends on a variety of factors, but is, as a vile, in the range of from 10:90 to 50:50, preferably from 20:80 to 30:70.

Customary dosages of the aminophenylamidines per day are from 1 to 100 mg/kg bodyweight, preferably from 2 to 60 mg/kg bodyweight.

Customary dosages of the depsipeptides per day are from 0.1 to 100 mg/kg bodyweight, preferably from 0.5 to 50 mg/kg bodyweight, especially preferably from 1 to 50 mg/kg bodyweight.

### EXAMPLES

### Example 1

### Liquid Formulation

Suspensions in 100 ml glycerol formal/glycerol polyethylene glycol ricinoleate (Cremophor® EL)/water in the mixing ratio 1:10 together with:
500 mg PF 1022, 1000 mg tribendimidine
500 mg PF 1022, 1500 mg tribendimidine
400 mg emodepside, 1000 mg tribendimidine
400 mg emodepside, 1500 mg tribendimidine
500 mg PF 1022, 1000 mg amidantel
500 mg PF 1022, 1500 mg amidantel
400 mg emodepside, 1000 mg amidantel
400 mg emodepside, 1500 mg amidantel

### Example 2

### Liquid Formulation

Suspensions in 100 ml Cremophor® EL/water in the mixing ratio 1:5 together with:
500 mg PF 1022, 1000 mg tribendimidine
500 mg PF 1022, 1500 mg tribendimidine
400 mg emodepside, 1000 mg tribendimidine
400 mg emodepside, 1500 mg tribendimidine
500 mg PF 1022, 1000 mg amidantel
500 mg PF 1022, 1500 mg amidantel
400 mg emodepside, 1000 mg amidantel
400 mg emodepside, 1500 mg amidantel

### Example 3

### Solid Formulation

The pulverulent active substance quantities detailed hereinbelow are filled into gelatin capsules:
100 mg PF 1022, 200 mg tribendimidine
100 mg PF 1022, 300 mg tribendimidine
80 mg emodepside, 250 mg tribendimidine
100 mg emodepside, 300 mg tribendimidine
100 mg PF 1022, 200 mg amidantel
100 mg PF 1022, 300 mg amidantel
150 mg emodepside, 400 mg amidantel
100 mg emodepside, 300 mg amidantel

### Biological Examples

### Synergistic Effect of the Combination of Amidines (for Example Amidantel, Bay d 9216) Together with Cyclic Octadepsipeptides (for Example PF1022A)

Test Method for In-Vitro Experiments with Nippostrongylus brasiliensis

Adult Nippostrongylus brasiliensis were isolated from the small intestine of female Wistar rats and transferred into 0.9% NaCl containing 20 μg/ml sisomycin and 2 μg/ml Canesten. The groups of worms (both sexes) were incubated in 1.0 ml of medium, which was also used for determining the acetylcholine esterase activity. The incubation and the enzyme assay were described in the paper by Rapson et al. (1987) Z. Parasitenkunde 73, 190-191. The compounds were dissolved in DMSO and added to the incubation medium so that final concentrations of 100, 10, 1, 0.1, 0.01, 0.001 and 0.0001 μg/ml were present. The controls only contained DMSO. The acetylcholine esterase activity in the incubation medium was determined in accordance with the above publication.

The efficacy was categorized by means of a scale 0-3, where 0=no activity (<50% enzyme inhibition); 1=weak activity; 2=good activity; 3=full activity

Amidantel

| | Conc. in ppm | 0 | 100 | 50 | 10 |
|---|---|---|---|---|---|
| PF1022A | 0 | X | 1 | 1 | 1 |
| | 0.1 | 2 | 2 | 2 | 2 |
| | 0.05 | 1 | 2 | 2 | 2 |
| | 0.01 | 1 | 2 | 2 | 2 |

Bay d 9216

| | Conc. in ppm | 0 | 10 | 5 | 1 |
|---|---|---|---|---|---|
| PF1022A | 0 | X | 1 | 1 | 1 |
| | 0.1 | 2 | 2 | 2 | 2 |
| | 0.05 | 1 | 2 | 2 | 2 |
| | 0.01 | 1 | 1 | 2 | 2 |

1. A formulation comprising anthelmintically active aminophenylamidine derivatives and cyclic depsipeptides.

2. The formulation of claim 1, in which the aminophenylamidine derivative is a compound of the formula (I), 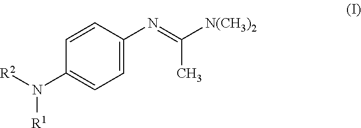
in which
R¹ is hydrogen or C₁₋₄-alkyl and
R² is hydrogen, —COO(C₁₋₄-alkyl), —CO(C₁₋₄-alkyl), —COCH₂(C₁₋₄-alkyl), —COCH₂O-phenyl or —CO-hetaryl, where hetaryl represents a 5- or 6-membered aromatic heterocycle with one or more hetero ring atoms selected from amongst O, N and S, or
R¹ and R² together represent the radical

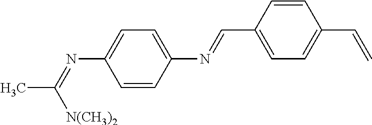

3. The formulation of claim 1, in which the aminophenylamidine derivative is a compound of the formula (I) in which
R¹ represents hydrogen,
R² represents hydrogen, —COCH₂(C₁₋₄-alkoxy) or together with R¹ represents the radical

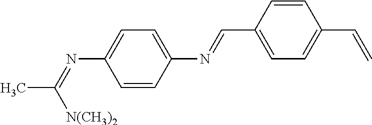

4. The formulation of claim 1, in which the aminophenylamidine derivative is amidantel, Bay d 9216 or tribendimidine.

5. The formulation of claim 1, in which the aminophenylamidine derivative is tribendimidine.

6. The formulation of claim 5, in which the cyclic depsipeptide is a 24-membered octacyclodepsipeptide.

7. The formulation of claim 6, in which the cyclic depsipeptide is emodepside.

8. The formulation of claim 6, in which the cyclic depsipeptide is PF 1022.

9. (canceled)

10. A method for controlling endoparasites in humans or animals comprising administering to an animal an effective amount of formulation comprising an anthelmintically active aminophenylamidine derivative and a cyclic octadepsipeptide.
